(19) United States
(12) Patent Application Publication
(10) Pub. No.: US 2003/0083226 A1
(43) Pub. Date: May 1, 2003

(54) Composition having reverse transcriptase inhibitor activity

(76) Inventor: Bruce Halstead, Grand Terrace, CA (US)

Correspondence Address: ROBERT D. FISH; RUTAN & TUCKER, LLP, P.O. BOX 1950, 611 ANTON BLVD., 14TH FLOOR, COSTA MESA, CA, 92628-1950, US

(21) Appl. No.: 10/159,747

(22) Filed: May 29, 2002

Related U.S. Application Data
(60) Provisional application No. 60/294,480, filed on May 30, 2001.

(51) Int. Cl. A61K 31/00

(52) U.S. Cl. 514/1

(57) ABSTRACT

A pharmacological composition comprises a synthetic reverse transcriptase inhibitor having a structure of a molecule that is present in a plant extract demonstrated to have an antiviral effect, wherein the molecule produces at least in part of the antiviral effect.

This application claims the benefit of U.S. provisional application No. 60/294,480 filed May 30, 2001, incorporated herein by reference in its entirety.

### FIELD OF THE INVENTION

The field of the invention is antiviral compositions.

### BACKGROUND OF THE INVENTION

Treatment of viral infections is frequently limited by the availability, tolerability, and cost of known and approved pharmacological agents. Moreover, even if one or more antiviral agents are relatively well tolerated (physically as well as financially), resistance to such agents tends to develop rather quickly. Therefore, there is a continuing need for novel antiviral compositions that are well tolerated and relatively inexpensive.

### DETAILED DESCRIPTION

The inventors recognized that various plant extracts exhibit significant antiviral activity and that reverse transcriptase inhibitors (RTI) may be isolated for such plant extracts. Moreover, the inventors contemplate that such RTIs can be characterized and/or synthesized de novo.

Particularly contemplated plants include Abies webbiana, Acacia spec., Acacia Arabia, Agrimonia eupatoria, Ajuga decumbens, Allium cepa, Allium sativum, Aloe vera, Altemanthera philoxeroides or sessiles, Ammi maius, Andographis paniculata, Apium graveolens, Apium leptophyllum, Arachis hypogaea, Arctium lappa, Amebia euhcroma, Asparagus racemosus, Astragalus spinosus, Astragalus lentingosis swainsonine, Buchenavia capita, Bryonia cretica ssp. Dioica, Bryonia angustifolia, Camellia theifera, Camellia sinensis, Cedrela toona, Chrysanthemum morifolium, Coffea arabica, Coptis chinesis, Coptis teetoides, Coptis japonica, Coraria nepalensis, Coriandrum sativum, Curcuma longa, Datura metel syn alba, Daucus carota, Echinacea angustiflora and purpurea, Echinacea simulata, Echinacea pallida, Epimedium grandiflorum, Epimedium sagittatum, Epimedium sinense, Epilobium angustifolium, Erigeron Canadensis, Eugenia or Syzigium claviflorum, Fagara xanthox, Foeniculum vulgarel, Gardenia coronaria, Gaultheria trichophylla, Glycine max, Glycyrrhiza labra, Gossypium herbaceum, Heracleum sphondylium, Hypericum perforatum, Hypericum japonicum, Hyssopus officinalis, Jasminum officinale, Lithospermum erythrorhizon, Lonicera japonica, Luffa luffa, Lycopus europaeus, Magnolia officinalis, Mallotus repandus, Mallotus philippinesis, Matricaria chamomil, Matricaria recutitia, Melissa parviflora, Melissa officinalis, Momordica balsamina, Momordica charantia, Narcissus tazetta, Narcissus pseudonarcissus, Oenthera rosea, Paeonia spec., Papaver somniferum, Perilla frutescens, Phyllanthus niruri, Pinus koraicenis, Pinus parviflora, Piper nirgum, Plumeria rubra, Polyantha suberosa, Prunella vulgaris, Prunus bakariensis, Prunus amygdalus, Psoralea corylifolia, Randia dunatorum, Raphanus sativus, Rheum palmatum, Rhus coriaria, Rhus chinesis, Ricinus communis, Rosmarinus officinalis, Salvia miltiorhiza and officinalis, Sambucus ebulus, Saussurea lappa, Scilla griffithii, Scutellaria baicalensis baiealein, Sedum sediforme, Senecio scandens, Senecio aereus, Skimmia laureola, Solarium niporum, Swertia franchetiana, Terminalia chebula, Terminalia catappa, Terminalia alata, Thula occidentalis, Trapalaponica spec., Trichosanthes dioica, Trichosanthes kirilowii, Urtica dioica, Viola yeodensis, Woodfordia fruticosa, Woodwardia spec., and Zanoxylum nitidum. However, in alternative aspects many plants other than the above-listed plants are also contemplated. In fact, all plants are contemplated that exhibit antiviral activity.

With respect to the identification of an RTI in contemplated plants, it should be appreciated that numerous assays are known in the art, and can readily be adapted to a screening process in which a fractions of a plant extract are screened for RTI activity. For example, U.S. Pat. No. 6,130,036 to Loeb et al. describes a high throughput assay system in which positive selective pressure is employed to select and/or identify an RTI. Once a fraction has been identified as having RTI activity, it is contemplated that further separation of the components in that fraction will eventually lead to an isolated (single or complex) compound.

It is still further contemplated that such isolated compounds may then be characterized using various forms of mass spectroscopy (e.g., ESMS, FAB-MS, GC-MS, etc.), UV-, IR-, and VIS-spectroscopy, atom absorption spectroscopy, various forms of NMR (¹H-NMR, ¹³C-NMR, NOE-NMR, etc.), or other analytical method. While not limiting to the inventive subject matter, it is preferred that such characterization methods will lead to a chemical structure of the RTI, which may be employed to synthesize the RTI de novo, or to modify the structure to arrive at an RTI with improved or altered physico-chemical properties.

Particularly contemplated modifications of isolated and characterized RTIs include increased specificity towards the viral polymerase over non-specific interactions with non-reverse transcriptase molecules in a cell or biological system, higher affinity of the modified RTI towards the reverse transcriptase, reduced toxicity, increased solubility, etc.

Consequently, it is contemplated that pharmacological composition comprises a synthetic reverse transcriptase inhibitor having a structure of a molecule that is present in a plant extract demonstrated to have an antiviral effect, wherein the molecule produces at least in part of the antiviral effect.

Thus, specific embodiments and applications of compositions having reverse transcriptase inhibitor activity have been disclosed. It should be apparent, however, to those skilled in the art that many more modifications besides those already described are possible without departing from the inventive concepts herein. The inventive subject matter, therefore, is not to be restricted except in the spirit of the appended contemplated claims. Moreover, in interpreting both the specification and the contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A pharmacological composition, comprising:
a synthetic reverse transcriptase inhibitor having a structure of a molecule that is present in a plant extract demonstrated to have an antiviral effect, wherein the molecule produces at least in part of the antiviral effect.

2. The composition of claim 1 wherein the synthetic reverse transcriptase inhibitor is further modified such that the modified synthetic reverse transcriptase inhibitor has a higher affinity to a reverse transcriptase that the molecule that is present in the plant.

3. The composition of claim 1 wherein the synthetic reverse transcriptase inhibitor is further modified such that the modified synthetic reverse transcriptase inhibitor has a lower toxicity than the molecule that is present in the plant.

4. The composition of claim 1 wherein the synthetic reverse transcriptase inhibitor is further modified such that the modified synthetic reverse transcriptase inhibitor has a higher specificity towards a reverse transcriptase than the molecule that is present in the plant.
